(19) United States
(12) Patent Application Publication
(10) Pub. No.: US 2026/0232597 A1
(43) Pub. Date: Aug. 13, 2026

(54) PHARMACEUTICAL COMPOSITION OF TRAMETINIB AND PROCESS OF PREPARATION THEREOF

(71) Applicant: ALEMBIC PHARMACEUTICALS LIMITED, Vadodara (IN)

(72) Inventors: Ashok Kumar SAHOO, Hyderabad (IN); Ashish Babulal RANE, Hyderabad (IN); Rahul Sudhakar DABRE, Hyderabad (IN)

(73) Assignee: ALEMBIC PHARMACEUTICALS LIMITED, Vadodara (IN)

(21) Appl. No.: 19/155,795

(22) PCT Filed: Feb. 12, 2024
(86) PCT No.: PCT/IB2024/051278

§ 371 Date: Aug. 12, 2025

(30) Foreign Application Priority Data
Feb. 13, 2023 (IN) 202321009528

(51) Int. Cl. A61K 9/20; A61K 9/00; A61K 9/14; A61K 31/519

(52) U.S. Cl. CPC A61K 9/2077; A61K 9/0053; A61K 9/146; A61K 9/2027; A61K 9/2054; A61K 31/519

(57) ABSTRACT

The present subject matter relates to a pharmaceutical composition comprising Trametinib or a pharmaceutically acceptable salt thereof and at least one pharmaceutical acceptable excipient. The present subject matter also relates to a solid oral pharmaceutical compositions comprising a solid dispersion of Trametinib or a pharmaceutically acceptable salt thereof and at least one pharmaceutical acceptable excipient. Furthermore, it also relates to methods for manufacturing such compositions, and uses thereof.

### TECHNICAL FIELD

The present subject matter relates to a pharmaceutical composition comprising Trametinib or a pharmaceutically acceptable salt thereof and at least one pharmaceutical acceptable excipient. The present subject matter also relates to a solid oral pharmaceutical composition comprising a solid dispersion of Trametinib or a pharmaceutically acceptable salt thereof and at least one pharmaceutical acceptable excipient. Furthermore, it also relates to methods for manufacturing such compositions, and uses thereof.

### BACKGROUND

Trametinib is a kinase inhibitor. It is chemically described as N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1 (2H)-yl}phenyl) acetamide. Its empirical formula is C₂₆H₂₃FIN₅O₄ and it has the following structural formula:

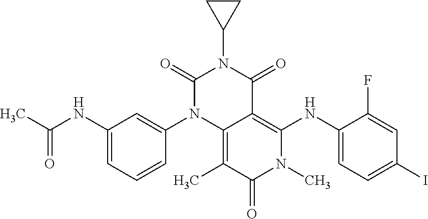 [SMILES: CC(=O)Nc1cccc(-n2c(=O)n(C3CC3)c(=O)c3c(Nc4ccc(I)cc4F)n(C)c(=O)c(C)c32)c1]

Trametinib is approved in the USA in the form of Trametinib dimethyl sulfoxide (DMSO) solvate form. It is approved as equivalent to 0.5, 1 & 2 mg base tablet under the brand name Mekinist® and is indicated for the treatment of patients with unresectable or metastatic melanoma with BRAF V600E or V600K mutations. Each 0.5 mg tablet contains 0.5635 mg Trametinib dimethyl sulfoxide equivalent to 0.5 mg of Trametinib unsolvated parent. Each 2 mg tablet contains 2.254 mg Trametinib dimethyl sulfoxide equivalent to 2 mg of Trametinib unsolvated parent.

Trametinib dimethyl sulfoxide is a white to almost white powder. It is practically insoluble in the pH range of 2 to 8 in aqueous media.

U.S. Pat. No. 7,378,423 (US'423 patent) discloses Trametinib and pharmaceutically acceptable salts and solvates thereof, as being useful as an inhibitor of MEK activity, particularly in treatment of cancer. US '423 patent further discloses Trametinib is in the form of a solvate selected from: hydrate, acetic acid, ethanol, nitromethane, chlorobenzene, 1-pentanol, isopropyl alcohol, ethylene glycol and 3-methyl-1-butanol and its preparation method thereof.

U.S. Pat. No. 8,580,304 (US'304 patent) discloses film coated oral pharmaceutical tablets comprising Trametinib dimethyl sulfoxide solvate (DMSO solvate) and one or more excipients, wherein the excipients are substantially free of water. US '304 patent further disclose that amount of Trametinib unsolvated (desolvated) form in the pharmaceutical tablet does not exceed about 20%, suitably does not exceed 15%, suitably does not exceed 10%, suitably does not exceed 5%, suitably does not exceed 2% as compare to Trametinib dimethyl sulfoxide solvate.

US'304 patent further disclose that Trametinib DMSO can undergo desolvation during handling and formulation resulting in unsolvated Trametinib. This unsolvated Trametinib is much less soluble than dimethyl sulfoxide solvate of Trametinib and negatively impacts its pharmacodynamics when released from a pharmaceutical composition. Additionally it was also found that unsolvated Trametinib can exhibit poor exposure and absorption upon in vivo administration.

US′304 also discloses that tablets wherein the amount of desolvated Trametinib does not exceed 20% and excipients are substantially free of water, provide an acceptable release/pharmacodynamic profile.

Still there is a need in the art for alternative pharmaceutical compositions of Trametinib or its pharmaceutically acceptable salt thereof with increased solubility and increased bioavailability which provide an acceptable release/pharmacodynamic profile.

Although use of DMSO solvate of Trametinib led to development of favorable formulation, DMSO solvates have many drawbacks like extra manufacturing step, use of solvent, stability issues and toxicity issues. Although DMSO solvate has drawbacks, the prior criticizes use of unsolvated form of Trametinib because of its properties and emphasizes on viable product with DMSO solvate.

During the course of development, it was found that contrary to conventional wisdom, it is possible to develop a pharmaceutical composition of unsolvated Trametinib with satisfactory, comparable or even improved properties than a composition with DMSO solvate. It was also found that such compositions can be prepared by conventional and well known processes.

During the course of development, it was found that solubility of unsolvated Trametinib may be improved through a solid dispersion (SD). It was also found that solid dispersion of unsolvated Trametinib may lead to improved solubility than DMSO solvate. It was also found that compositions containing solid dispersion of unsolvated Trametinib may exhibit higher dissolution than compositions of DMSO solvate of Trametinib.

The present subject matter relates to a pharmaceutical composition comprising Trametinib or a pharmaceutically acceptable salt thereof and at least one pharmaceutical acceptable excipient. The present subject matter also relates to a solid oral pharmaceutical composition comprising a solid dispersion of Trametinib or a pharmaceutically acceptable salt thereof and at least one pharmaceutical acceptable excipient. Furthermore, it also relates to methods for manufacturing such compositions, and uses thereof.

In an embodiment, the present subject matter relates to pharmaceutical composition comprising unsolvated Trametinib and at least one pharmaceutical acceptable excipient. The present subject matter also relates to a solid oral pharmaceutical compositions comprising a solid dispersion of unsolvated Trametinib and at least one pharmaceutical acceptable excipient. Furthermore, it also relates to methods for manufacturing such compositions exhibiting enhanced solubility and acceptable release/pharmacodynamic profile.

In an embodiment, the present subject matter relates to a solid dispersion of unsolvated Trametinib comprising at least one polymer. Furthermore, it also relates to methods for manufacturing such compositions, and to their use.

Solid dispersion of unsolvated Trametinib may be prepared by any known means, including non-limiting examples such as, solvent evaporation, spray-drying, top-spray granulation, hot melt extrusion, precipitation from solution on addition of a non-solvent, use of hot melt granulation technique, use of lyophilization technology or freeze drying, complexation with β cyclodextrin, use of anti-solvent precipitation, supercritical fluid (SCF) technology.

It was also observed that solid dispersion of unsolvated Trametinib as per the present disclosure provides stable composition with enhanced dissolution profile.

In an embodiment, the present subject matter relates to a pharmaceutical composition comprising solid dispersion of unsolvated Trametinib. The present subject matter also relates to a solid dispersion of unsolvated Trametinib comprising at least one polymer. Furthermore, it also relates to methods for manufacturing such compositions, and to their use.

### SUMMARY OF THE SUBJECT MATTER

The present subject matter relates to a pharmaceutical composition comprising Trametinib or a pharmaceutically acceptable salt thereof and at least one pharmaceutical acceptable excipient. The present subject matter also relates to a solid oral pharmaceutical compositions comprising a solid dispersion of Trametinib or a pharmaceutically acceptable salt thereof and at least one pharmaceutical acceptable excipient. Furthermore, it also relates to methods for manufacturing such compositions, and uses thereof.

In an embodiment, the present subject matter relates to a solid oral pharmaceutical composition comprising unsolvated Trametinib and at least one pharmaceutical acceptable excipient. The present subject matter also relates to a solid oral pharmaceutical compositions comprising a solid dispersion of unsolvated Trametinib and at least one pharmaceutical acceptable excipient. Furthermore, it also relates to methods for manufacturing such compositions which enhance solubility and provide an acceptable release/pharmacodynamic profile.

An aspect of the present subject matter provides a solid oral pharmaceutical composition comprising a solid dispersion of unsolvated Trametinib and at least one polymer.

The ratio of the polymer to Trametinib is not particularly limited, so long as Trametinib can be formed the solid dispersion. In some embodiments, the ratio of the polymer to Trametinib is not particularly limited, so long as Trametinib can be an amorphous state. The ratio of the polymer is specifically 0.5 to 50 parts by weight in some embodiments, 0.5 to 25 parts by weight in some embodiments, 0.5 to 10 parts by weight in some embodiments 0.5 to 6 parts by weight in some embodiments, 1 to 3 parts by weight in some embodiments, 2 to 3 parts by weight in some embodiments, 3 to 5 parts by weight in some embodiments, and 5 parts by weight in some embodiments, with respect to 1 part by weight of Trametinib. In an embodiment, the ratio of polymer is 6 parts to 1 part by weight of Trametinib.

Another aspect of the present subject matter provides a manufacturing process to prepare solid dispersion comprising unsolvated Trametinib and at least one polymer. Such a solid dispersion can be prepared by conventional processes known in the art such as solvent evaporation, spray-drying, top-spray granulation, Hot Melt Extrusion (HME), precipitation from solution on addition of a non-solvent, use of hot melt granulation technique, use of lyophilization technology or freeze drying, complexation with β cyclodextrin, use of anti-solvent precipitation, supercritical fluid (SCF) technology.

Another aspect of the present subject matter provides a manufacturing process to prepare a pharmaceutical composition comprising a solid dispersion of Trametinib and at least one polymer, which is further processed to prepare dosage form amenable to be taken by patient. The pharmaceutical composition in this context may be any conventional dosage form known in the art such as tablet, capsule, granules and pellets. The preferred dosage forms are tablet and capsule.

### DETAILED DESCRIPTION OF THE SUBJECT MATTER

As used herein, “a” or “an” means one or more unless otherwise specified.

The term “composition” is intended to encompass a product comprising the specified ingredients in the specified amounts, as well as any product which results, directly or indirectly, from combinations of the specified ingredients in the specified amounts.

The term “Trametinib” as used herein according to the present subject matter is an active pharmaceutical ingredient and includes Trametinib in the form of a free base or an unsolvated form.

The term “amorphous” as used herein includes, but is not limited to, the substance substantially lacking a crystalline structure.

The term “crystalline” as used herein includes, but is not limited to, the substance having a specific crystalline structure.

The term “stable” and “stability” as used herein refers to both the physical form and the chemical purity of the active pharmaceutical ingredient or the pharmaceutical active dosage form including non-limiting examples of tablet, capsules.

The term “carrier” as used herein refers to a pharmaceutically acceptable excipient which can be utilized to prepare compositions of the present subject matter with enhanced dissolution and act as carrier for the active pharma ingredient.

The term “substantially in amorphous form” as used herein refers to amorphous form containing less than 20% crystallinity, more preferably less than 10% crystallinity, less than 5% of crystallinity and more preferably less than 2% of crystallinity.

The term “substantially in crystalline form” as used herein refers to a crystalline form of more than 50% crystallinity, more preferably more than 70% crystallinity, more preferably more than 90% crystallinity and more preferably more than 98% of crystallinity.

In one embodiment, Trametinib is present in the form of “unsolvated form”, which is also known as non-solvated form. (i.e. substantially free of water or other organic solvents as part of its structure)

The unsolvated form may contain less than 1.5% w/w of water or other organic solvents. Preferably, it contains less than 0.8% w/w, more preferably less than 0.6% w/w, even more preferably less than 0.4% w/w, such as less than 0.2% w/w or less than 0.1% w/w of water or other organic solvents. Most preferably, the active ingredient is substantially free of water or other organic solvents. In this respect, all amounts in % w/w are based on the total weight of Trametinib in its salt free and non-solvate form present in the composition.

During the course of development, it was found that contrary to conventional wisdom, it is possible to have a pharmaceutical composition with satisfactory dissolution, pharmacokinetics and therefore pharmacodynamics thereby doing away with use of any solvate. One of the means to make use of unsolvated Trametinib in the pharmaceutical composition is through a solid dispersion of unsolvated Trametinib. It was also found that compositions containing solid dispersion of unsolvated Trametinib exhibits higher dissolution than compositions of DMSO solvate of Trametinib.

It was also observed that solid dispersion of unsolvated Trametinib as per the present disclosure provides stable composition with enhanced solubility and dissolution profile.

The present subject matter relates to a solid oral pharmaceutical composition comprising unsolvated Trametinib and at least one pharmaceutical acceptable excipient. The present subject matter also relates to a solid oral pharmaceutical compositions comprising a solid dispersion of unsolvated Trametinib and at least one pharmaceutical acceptable excipient. Furthermore, it also relates to methods for manufacturing such compositions which enhance solubility and provide an acceptable release/pharmacodynamic profile.

In another embodiment, the present subject matter also relates to a solid dispersion of unsolvated Trametinib comprising at least one polymer. Furthermore, it also relates to methods for manufacturing such compositions, and to their use.

A solid dispersion of unsolvated Trametinib may be prepared by any known means, including non-limiting examples such as solvent evaporation, spray-drying, top-spray granulation, hot melt extrusion, precipitation from solution on addition of a non-solvent use of hot melt granulation technique, use of lyophilization technology or freeze drying, complexation with β cyclodextrin, use of anti-solvent precipitation, supercritical fluid (SCF) technology.

In one embodiment, Trametinib present in the pharmaceutical composition is substantially in the form of “unsolvated form”. In an aspect, the solvated compound in the pharmaceutical composition does not exceed about 20%, suitably the amount of solvated compound does not exceed about 15%, suitably the amount of solvated compound does not exceed about 10%, suitably the amount of solvated compound does not exceed about 5% and suitably the amount of solvated compound does not exceed about 2%.

In an embodiment of the present subject matter, a solid oral pharmaceutical composition comprises unsolvated Trametinib in the form of ‘substantially in amorphous form’.

In an embodiment of the present subject matter, a solid oral pharmaceutical composition comprises unsolvated Trametinib in the form of ‘substantially in crystalline form’.

In an embodiment, the present subject matter also relates to a solid oral pharmaceutical composition comprising solid dispersion of unsolvated Trametinib, wherein unsolvated Trametinib is substantially in amorphous form.

In an embodiment, the present subject matter also relates to a solid oral pharmaceutical composition comprising solid dispersion of unsolvated Trametinib, wherein unsolvated Trametinib is substantially in crystalline form.

In an embodiment, the present subject matter also relates to a solid oral pharmaceutical composition comprising solid dispersion of unsolvated Trametinib, wherein unsolvated Trametinib is in the form of a mixture of amorphous form and crystalline form.

Additional embodiment of the present subject matter relates to a solid oral pharmaceutical composition comprising solid dispersion of unsolvated Trametinib wherein Trametinib is retained in substantially amorphous form. Additional embodiment of the present subject matter relates to a pharmaceutical composition comprising solid dispersion of unsolvated Trametinib, Trametinib is retained in substantially amorphous form the composition is substantially free of crystalline forms of Trametinib.

Another aspect of the present subject matter provides a process for preparation of a solid oral pharmaceutical composition prepared by solid dispersion of unsolvated Trametinib and polymer by any known means, including non-limiting examples such as solvent evaporation, spray-drying, top-spray granulation, hot melt extrusion, precipitation from solution on addition of a non-solvent, use of hot melt granulation technique, use of lyophilization technology or freeze drying, complexation with β cyclodextrin, use of anti-solvent precipitation, supercritical fluid (SCF) technology.

Another aspect of the present subject matter provides a process for preparation of a solid oral pharmaceutical composition wherein solid dispersion is further processed into a pharmaceutical composition. Like dry granulation of solid dispersion with excipients or wet granulation with excipients or direct mixing with excipients etc. to make a pharmaceutical composition e.g. a tablet.

The spray drying process according to the subject matter refers to the process where the amorphous dispersion is formed by dispersing or dissolving the drug substance, polymer and/or other excipients in a suitable solvent and/or mixture of solvents to form a feed solution, pumping the feed solution through an atomizer into a drying chamber, and removing the solvent to form the amorphous solid dispersion powder in the drying chamber. The said solid dispersion can be mixed/granulated with excipients to form the final formulation.

Top spray granulation according to the subject matter refers to the process where amorphous dispersion is formed by dissolving the drug substance and/or polymer and/or other excipients in suitable solvent and/mixture of solvents and spraying the resultant solution onto a fluidized bed substrate (or “stabilizer and/or other excipients”). Typically the spraying is performed using an atomizing nozzle. The substrate is maintained in a fluidized state in a fluidized bed while the granulation binder is sprayed onto the substrate particles. In one aspect, a plurality of substrates is used in the process of the invention. Simultaneously, the solvent is evaporated while maintaining the resulting solid dispersion granules in the fluidized state. The fluidization of the substrate is performed in top-spray apparatus known in the art used to fluidize particles and form a fluidized bed. In the method of the invention the solvent is at least partially or completely evaporated to form the amorphous dispersion granules of the invention. The said solid dispersion can be mixed/granulated with extra granular excipients to form the final formulation.

The hot melt extrusion process according to the subject matter refers to the process where the dry mix for extrusion is prepared containing pharmaceutical active ingredient, polymer and/or other excipients which is processed in a hot melt extruder and lubrication is performed and the lubricated blend is further compressed into tablets and coated.

In one embodiment, the pharmaceutical composition comprising solid dispersion of unsolvated Trametinib is prepared, which comprises the steps as:
a) providing a solution of unsolvated Trametinib and at least one polymer and/or at least one pharmaceutically acceptable excipient in an inert solvent or mixture of solvents;
b) removing the solvent from the solution obtained in step a), and
c) isolating the solid dispersion of unsolvated Trametinib.
d) mixing solid dispersion of unsolvated Trametinib with extragranular excipients and processing the resultant mixture with roller compaction to obtain granules,
e) lubricating the granules obtained in step e) with extragranular material and formulating the mixture to obtain a suitable dosage form.

In one embodiment, the pharmaceutical composition comprising solid dispersion of unsolvated Trametinib is prepared, which comprises the steps as:
a) providing a solution of unsolvated Trametinib and at least one polymer and/or at least one pharmaceutically acceptable excipient in an inert solvent or mixture of solvents;
b) spraying the resultant solution on to the fluidized bed substrate.
c) simultaneous evaporation or removal of the solvent from the solution obtained in step a), and
d) isolating the solid dispersion of unsolvated Trametinib.
e) mixing solid dispersion of unsolvated Trametinib with extragranular excipients and processing the resultant mixture with roller compaction to obtain granules,
f) lubricating the granules obtained in step e) with extragranular material and formulating the mixture to obtain a suitable dosage form.

The ratio of the polymer to Trametinib is not particularly limited, so long as Trametinib can be formed the solid dispersion. In some embodiments, the ratio of the polymer to Trametinib is not particularly limited, so long as Trametinib can be an amorphous state. The ratio of the polymer is specifically 0.5 to 50 parts by weight in some embodiments, 0.5 to 25 parts by weight in some embodiments, 0.5 to 10 parts by weight in some embodiments 0.5 to 6 parts by weight in some embodiments, 1 to 3 parts by weight in some embodiments, 2 to 3 parts by weight in some embodiments, 3 to 5 parts by weight in some embodiments, and 5 parts by weight in some embodiments, with respect to 1 part by weight of Trametinib. In an embodiment, the ratio of polymer is 6 parts to 1 part by weight of Trametinib.

In further embodiment, solid dispersion retains Trametinib in amorphous form as characterized by e.g. Powder X-Ray Diffraction (PXRD) analysis. In further embodiment, PXRD spectra so obtained are substantially devoid of crystalline peaks.

In one embodiment, the pharmaceutical acceptable composition of the present application are stable for at least one month under packed condition. In embodiments, the pharmaceutical acceptable composition of the present application are stable for at least two months under packed condition. In embodiments, the pharmaceutical acceptable composition of the present application is stable for at least six months under packed condition.

In one embodiment, the pharmaceutical acceptable composition of the present application exhibits enhanced solubility with improved dissolution and bioavailability.

Another aspect of the present subject matter provides a pharmaceutical composition comprising,
(a) Unsolvated Trametinib,
(b) one or more pharmaceutically acceptable polymer,
(c) one or more pharmaceutically acceptable surfactant
(d) one or more pharmaceutically acceptable diluent,
(e) one or more pharmaceutically acceptable disintegrant,
(f) one or more pharmaceutically acceptable lubricant.

Further the present subject matter provides a pharmaceutical composition comprising,
(a) Unsolvated Trametinib,
(b) one or more pharmaceutically acceptable polymer,
(c) one or more pharmaceutically acceptable surfactant,
(d) one or more pharmaceutically acceptable binder,
(e) one or more pharmaceutically acceptable diluent,
(f) one or more pharmaceutically acceptable disintegrant.
(g) one or more pharmaceutically acceptable lubricant.

In one embodiment the present subject matter relates to a solid oral pharmaceutical composition comprising Trametinib or a pharmaceutically acceptable salt thereof and at least one of the following excipients:
(a) 0.1-25% w/w Trametinib or a pharmaceutically acceptable salt thereof,
(b) 0.1-50% w/w polymer(s),
(c) 0-2% w/w surfactant(s),
(d) 0-50% w/w Binder(s),
(e) 0.1-85% w/w Diluent(s),
(f) 0.1-20% w/w Disintegrant(s),
(g) 0.5-4% w/w Lubricant(s),
(h) 0.1-3% w/w Glidant(s).

Further the present subject matter provides a pharmaceutical composition comprising,
(a) Unsolvated Trametinib,
(b) one or more pharmaceutically acceptable polymer,
(c) one or more pharmaceutically acceptable surfactant
(d) one or more pharmaceutically acceptable diluent,
(e) one or more pharmaceutically acceptable disintegrant,
(f) one or more pharmaceutically acceptable lubricant.
wherein a solid dispersion of Trametinib and polymer is first prepared by solvent evaporation process.

Further the present subject matter provides a pharmaceutical composition comprising,
(a) Unsolvated Trametinib,
(b) one or more pharmaceutically acceptable polymer,
(c) one or more pharmaceutically acceptable surfactant
(d) one or more pharmaceutically acceptable diluent,
(e) one or more pharmaceutically acceptable disintegrant,
(f) one or more pharmaceutically acceptable lubricant,
wherein a solid dispersion of Trametinib and polymer is first prepared by HME.

Further the present subject matter provides a pharmaceutical composition comprising,
(a) Unsolvated Trametinib,
(b) one or more pharmaceutically acceptable polymer,
(c) one or more pharmaceutically acceptable surfactant
(d) one or more pharmaceutically acceptable diluent,
(e) one or more pharmaceutically acceptable disintegrant,
(f) one or more pharmaceutically acceptable lubricant.
wherein a solid dispersion of Trametinib and polymer is first prepared by top spray granulation process.

Further the present subject matter provides a pharmaceutical composition comprising,
(a) Unsolvated Trametinib
(b) one or more pharmaceutically acceptable polymer,
(c) one or more pharmaceutically acceptable surfactant
(d) one or more pharmaceutically acceptable diluent,
(e) one or more pharmaceutically acceptable disintegrant,
(f) one or more pharmaceutically acceptable lubricant.
wherein a solid dispersion of Trametinib and polymer is first prepared by spray drying process.

In this specification the terms “polymer” and “polymers” are intended to be interpreted in the context of pharmaceutical formulation science. Suitable polymer according to the present subject matter can be selected form the group of, but not limited to polyvinyl pyrrolidone and its derivatives thereof, povidone K-30, Povidone K-60, Povidone K-90, Kollidone K30, Vinylpyrrolidone-vinyl acetate copolymer, Kollidone VA64, Plasdone, Copovidones, Plasdone S630, polyoxyethylene-polyoxypropylene copolymers (Poloxamer), poloxamer 188 (Kolliphor P 188), polyvinylpyrrolidone vinyl acetate, polyvinylacetal diethylaminoacetate (AEA®), polyvinyl acetate phthalate, polysorbate 80, polyoxyethylene (40) stearate, polyethylene glycol monomethyl ether, polyethylene glycol, methylcellulose, methacrylic acid copolymer (Eudragit or Eudragit-EPO), hydroxypropylmethyl cellulose (HPMC), hydroxypropyl cellulose (HPC), hydroxypropylmethyl cellulose phthalate, hydroxypropylmethyl cellulose acetate succinate (HPMC-AS), hydroxypropylmethyl cellulose, hydroxypropyl cellulose SSL (HPC-SSL), hydroxypropyl cellulose SL (HPC-SL), hydroxypropyl cellulose L (HPC-L), hydroxyethyl cellulose, Soluplus® (polyvinyl caprolactam-polyvinyl acetate-polyethylene glycol graft copolymer (PCL-PVAc-PEG), Gelucire, ethyl cellulose, D-alpha-tocopheryl polyethylene glycol 1000 succinate, cellulose acetate phthalate, carboxymethylethylcellulose, cyclodextrin, Hydroxypropyl-beta-cyclodextrin (HPBCD), Sulfobutyl ether β-cyclodextrin (SBCD) Polyoxyl castor oil and the like.

The term “diluent” is intended to be interpreted in the context of pharmaceutical formulation science. Suitable diluents according to the present subject matter can be selected form the group of, but not limited to microcrystalline cellulose (MCC), pregelatinized starch, sugar and sugar alcohol such as mannitol, lactose, fructose, isomalt, lactitol, maltitol, sorbitol, erythritol, maltose, polydextrose, sucrose, trehalose and xylitol; calcium carbonate, dicalcium phosphate, calcium sulfate, cellulose acetate, ethylcellulose, inulin, magnesium carbonate, magnesium oxide, maltodextrin, sodium bicarbonate, sodium carbonate and sodium chloride.

The terms “surfactant” and “surfactants” are intended to be interpreted in the context of pharmaceutical formulation science. Suitable pharmaceutical surfactant according to the present subject matter can be selected form but not limited to sodium lauryl sulfate (SLS), poloxamers, polysorbate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like.

The term “binder” is intended to be interpreted in the context of pharmaceutical formulation science. Suitable binder according to the present subject matter can be selected form the group of, but not limited to povidone, copovidone pregelatinized starch, cellulose, methyl cellulose, ethyl cellulose, cellulose derivatives (e.g., Hydroxypropyl methyl cellulose (HPMC), hydroxypropyl cellulose (HPC)), and polyethylene glycol.

The terms “disintegrant” and “disintegrants” are intended to be interpreted in the context of pharmaceutical formulation science. Suitable disintegrants according to the present subject matter can be selected form but not limited to croscarmellose sodium (CCS), sodium starch glycolate (SSG), pregelatinized starch, alginic acid, sodium alginate, carboxymethylcellulose calcium, chitosan, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, and starch.

The terms “lubricant” and “lubricants” are intended to be interpreted in the context of pharmaceutical formulation science. Suitable pharmaceutical lubricant according to the present subject matter can be selected form but not limited to magnesium, aluminium, zinc or calcium stearate, glyceryl behenate, glyceryl dibehenate, glyceryl monostearate, glyceryl palmitostearate, a mixture of behenate esters of glycerine (e.g. a mixture of glyceryl bihenehate, tribehenin and glyceryl behenate), magnesium stearate, myristic acid, palmitic acid, stearic acid, talc, tribehenin, sodium stearyl fumarate (SSF) and sucrose stearate.

The terms glidants are frequently used in tablet formulations to improve flow. Suitable glidants according to the present subject matter can be selected from but not limited to magnesium oxide, silicon dioxide, colloidal silicon dioxide, pyrogenic silica, talc and combinations thereof. In some embodiments, the glidant comprises silicon dioxide.

Still another embodiment of the present subject matter provides a composition wherein the organic solvent used in preparation of pharmaceutical composition is selected from isopropyl alcohol, ethanol, methanol, acetone, dichloromethane (DCM), acetonitrile, petroleum ether or their mixture thereof,

Another aspect of the present subject matter provides a process for preparation of a solid oral pharmaceutical composition wherein solid dispersion is further processed into a pharmaceutical composition. Like dry granulation of solid dispersion with excipients or wet granulation with excipients or direct mixing with excipients etc. to make a pharmaceutical composition e.g. a tablet.

As used herein, the word “comprising” describes components that must be present, but leaves open the possibility that other unspecified components may also be present within the scope of the relevant term.

In one aspect there is provided a pharmaceutical tablet comprising the pharmaceutical composition as defined herein.

In one embodiment there is provided a pharmaceutical tablet comprising a pharmaceutical composition in the form of tablet core, wherein the tablet core is coated with a layer of coating. In one embodiment the coating is a film coating.

When the tablet has a film coating, the film coating may be applied using conventional methods. A coating can be used to provide protection against, for example, moisture ingress or degradation by light, to color the formulation, or to modify or control the release of Unsolvated Trametinib from the formulation.

In one embodiment, the pharmaceutical composition is a pharmaceutical tablet composition. In one embodiment, the pharmaceutical composition is a pharmaceutical tablet composition suitable for oral administration.

In one embodiment, the pharmaceutical composition is a pharmaceutical tablet composition suitable for oral administration for the treatment of various melanoma with BRAF V600E or V600K mutations, non-small lung cancer, anaplastic thyroid cancer, in patient in need thereof by administering compositions of the present subject matter.

In one embodiment, the pharmaceutical tablet comprises 0.5 mg, 1 mg & 2 mg unsolvated Trametinib.

Solid dispersions of the present subject matter also include the solid dispersions obtained by combining unsolvated Trametinib with a suitable non-polymeric excipient by employing techniques known in the art or procedures described or exemplified in any aspect of the instant application.

Embodiments provided herein may be more fully understood by reference to the following examples. These examples are meant to be illustrative of pharmaceutical composition and dosage form provided herein, but is not in any way limiting.

### Example 1

TABLE 1
Saturation solubility of Trametinib DMSO solvate & Trametinib

| | Saturation Solubility | | | | | | | |
|---|---|---|---|---|---|---|---|---|
| | Trametinib DMSO | | | | Trametinib | | | |
| | Solubility at 37° C._24 hrs | | | | Solubility at 37° C._24 hrs | | | |
| | Average | mg/ | mg/ | mg/ | Average | mg/ | mg/ | mg/ |
| | solubility | 250 | 500 | 900 | solubility | 250 | 500 | 900 |
| Media Name | mg/mL | mL | mL | mL | mg/mL | mL | mL | mL |
| 0.1N HCl | 0.00 | 0.8 | 2 | 3 | 0.02 | 4.2 | 8 | 15 |
| pH 4.5 Acetate | 0.00 | 0.0 | 0 | 0 | 0.01 | 2.5 | 5 | 9 |
| Buffer | | | | | | | | |
| pH 6.8 | 0.00 | 0.0 | 0 | 0 | 0.02 | 4.2 | 8 | 15 |
| Phosphate | | | | | | | | |
| Buffer | | | | | | | | |
| pH 7.0 | 0.00 | 0.0 | 0 | 0 | 0.00 | 0.0 | 0 | 0 |
| Phosphate | | | | | | | | |
| Buffer | | | | | | | | |
| 0.1N HCl + | 0.05 | 11.7 | 23 | 42 | 0.07 | 17 | 33 | 60 |
| 0.75% SDS | | | | | | | | |
| pH 4.5 Acetate | 0.24 | 60.0 | 120 | 216 | 0.10 | 24 | 48 | 87 |
| Buffer + | | | | | | | | |
| 0.75% SDS | | | | | | | | |
| pH 6.8 | 0.07 | 18.3 | 37 | 66 | 0.01 | 3 | 5 | 9 |
| Phosphate | | | | | | | | |
| Buffer + | | | | | | | | |
| 0.75% SDS | | | | | | | | |
| pH 7.0 | 0.07 | 18.3 | 37 | 66 | 0.01 | 3 | 5 | 9 |
| Phosphate | | | | | | | | |
| Buffer + | | | | | | | | |
| 0.75% SDS | | | | | | | | |

### Procedure:

Solubility study of drug substance was carried out in different dissolution media. The saturated solutions of drug substance were prepared in the recommended dissolution media. Initial pH of the saturated solutions was noted. Then the solutions were allowed to shake for 24 hours in the incubator shaker at 150 RPM. The temperature was kept around 37° C.±0.5° C. After 24 hours of shaking, pH of saturated solutions was noted. Further dilutions of these solutions were made as per the standard concentration. Analysis was carried out using HPLC technique. The sample was prepared in triplicates for saturation solubility determination.

### Example 2

TABLE 2
Solid dispersion of Trametinib (unsolvated) with polymer

| Sr. | | Carrier | | | | |
|---|---|---|---|---|---|---|
| No. | API | (Polymer) | API:Polymer | Solvent | PXRPD | Process |
| TA0 | Trametinib | — | — | — | Crystalline | — |
| TA1 | Trametinib | — | — | — | — | — |
| | DMSO | | | | | |
| TA2 | Trametinib | — | — | — | — | — |
| | acetic acid | | | | | |
| | solvate | | | | | |
| TA3 | Trametinib | Kollidone VA64 | 1:5 | DCM, | Amorphous | Solvent |
| | | | | Methanol | | evaporation |
| TA4 | Trametinib | HPBCD | 1:5 | DCM, | Amorphous | |
| | | | | Methanol | | |
| TA5 | Trametinib | Kollidone K30 | 1:5 | DCM, | Amorphous | |
| | | | | Methanol | | |
| TA6 | Trametinib | Plasdone S630 | 1:5 | DCM, | Amorphous | |
| | | | | Methanol | | |
| TA7 | Trametinib | Plasdone S630 | 1:10 | DCM, | Amorphous | |
| | | | | Methanol | | |
| TA8 | Trametinib | Eudragit EPO | 1:5 | DCM, | Amorphous | |
| | | | | Methanol | | |
| TA9 | Trametinib | Eudragit EPO | 1:10 | DCM, | Amorphous | |
| | | | | Methanol | | |
| TA10 | Trametinib | Kolliphor P 188 | 1:5 | DCM, | Amorphous | |
| | | | | Methanol | | |
| TA11 | Trametinib | Gellucire 48/16 | 1:20 | DCM, | Crystalline | |
| | | | | Methanol | | |
| TA12 | Trametinib | Polysorbate 80 | 1:20 | DCM, | Crystalline | |
| | | | | Methanol | | |
| TA13 | Trametinib | Kollidone K30 | 1:6 | DCM, | Amorphous | |
| | | | | Methanol | | |
| TA14 | Trametinib | Kollidone VA64 | 1:10 | NA | Crystalline | HME |

### The Procedure for Example 2 (TA3 to TA13) Comprises the Steps:

1. Unsolvated Trametinib and polymer was dissolved in a mixture of non-aqueous solvents to form a clear solution.
2. Solvent was removed from the solution which was obtained in step (1) with desired process parameters.
3. The solid dispersion powder which was obtained in step 2 was isolated and used for pharmaceutical composition preparation

### The Procedure for Example 2 (TA14) Comprises the Steps:

1. A solid dry mix of unsolvated Trametinib, polymer and/or pharmaceutically acceptable excipient was prepared.
2. The dry mix which was obtained in step 1) was processed through hot melt extruder.
3. The milled solid dispersion material which was obtained in step 2 was isolated and used for pharmaceutical composition preparation.

### Example 3

TABLE 3
Results of dissolution studies for Example 2 (TA0 to TA14)

| Sr.No. | API (A) | Polymer (P) | A:P | % DR | | | | | | | | |
|---|---|---|---|---|---|---|---|---|---|---|---|---|
| 4.5 Acetate Buffer + 0.75% SLS | | | | Time points (mins) | | | | | | | | |
| (Type II, 500 mL, 60 RPM) | | | | 5 | 10 | 15 | 20 | 30 | 45 | 60 | 90 | 120 |
| TA0 | Trametinib | NA | NA | 9 | 11 | 12 | 13 | 14 | 16 | 17 | 19 | 21 |
| TA1 | Trametinib | NA | NA | 27 | 34 | 38 | 39 | 41 | 42 | 43 | 44 | 44 |
| | DMSO | | | | | | | | | | | |
| TA2 | Trametinib | NA | NA | 2 | 3 | 5 | 7 | 9 | 13 | 16 | 23 | 29 |
| | acetic acid | | | | | | | | | | | |
| | solvate | | | | | | | | | | | |
| TA3 | Trametinib | Kollidone | 1.5 | 96 | 96 | 97 | 98 | 98 | 99 | 99 | 100 | 101 |
| | | VA64 | | | | | | | | | | |
| TA4 | Trametinib | HPBCD | 1.5 | 39 | 53 | 62 | 70 | 81 | 86 | 90 | 94 | 97 |
| TA5 | Trametinib | Kollidone | 1.5 | 91 | 94 | 95 | 95 | 95 | 96 | 96 | 97 | 97 |
| | | K30 LP | | | | | | | | | | |
| TA6 | Trametinib | Plasdone | 1.5 | 81 | 88 | 92 | 94 | 95 | 96 | 97 | 98 | 99 |
| | | S630 | | | | | | | | | | |
| TA7 | Trametinib | Plasdone | 1.10 | 94 | 95 | 97 | 98 | 98 | 99 | 99 | 99 | 99 |
| | | S630 | | | | | | | | | | |
| TA8 | Trametinib | Eudragit | 1.5 | 37 | 56 | 70 | 78 | 85 | 91 | 93 | 96 | 99 |
| | | EPO | | | | | | | | | | |
| TA9 | Trametinib | Eudragit | 1.10 | 53 | 74 | 79 | 83 | 87 | 90 | 93 | 95 | 97 |
| | | EPO | | | | | | | | | | |
| TA10 | Trametinib | Kolliphor P | 1.5 | 95 | 95 | 98 | 100 | 100 | 102 | 102 | 104 | 104 |
| | | 188 Micro | | | | | | | | | | |
| TA11 | Trametinib | Gellucire | 1.20 | 75 | 79 | 81 | 81 | 86 | 92 | 98 | 102 | 103 |
| | | 48/16 | | | | | | | | | | |
| TA12 | Trametinib | Polysorbate | 1.20 | 36 | 40 | 43 | 45 | 50 | 57 | 62 | 70 | 76 |
| | | 80 | | | | | | | | | | |
| TA13 | Trametinib | Kollidone | 1.6 | 77 | 81 | 83 | 85 | 86 | 88 | 88 | 89 | 90 |
| | | K30 | | | | | | | | | | |
| TA14 | Trametinib | Kollidone | 1.10 | 108 | 109 | 109 | 109 | 109 | 110 | 111 | 112 | 112 |
| | | VA64 | | | | | | | | | | |

### Example 4

TABLE 4
Pharmaceutical composition of Trametinib

| | | % of ingredient |
|---|---|---|
| Sr. | | by weight of |
| No. | Ingredients | core tablet |
| 1 | Trametinib | 0.1-25% |
| 2 | Polymer (s) (e.g. Povidone, Copovidone , HPMC | 0.1-50% |
| | HPC, Eudragit, Poloxamer) | |
| 3 | Diluent(s) (e.g. Microcrystalline cellulose, | 0.1-85% |
| | Mannitol, pregelatinized starch, etc.) | |
| 4 | Disintegrant(s) (e.g. croscarmellose sodium, etc.) | 0.1-20% |
| 5 | Optionally Binder(s) (e.g. povidone, etc.) | 0-50% |
| 6 | Optionally surfactant (s) (e.g. SLS, etc.) | 0-2% |
| 7 | Lubricant(s) (e.g. Magnesium stearate, talc, etc.) | 0.5-4% |
| 8 | Glidant(s) (e.g. colloidal silicon dioxide, etc.) | 0.1-3% |
| 9 | Optionally Film Coating | q.s |

### Example 5 and 6

TABLE 5
Pharmaceutical composition Trametinib

Example

| | | 5 | 6 |
|---|---|---|---|
| | Ingredient Name | % w/w | % w/w |
| 1 | Trametinib | 1.11 | 1.29 |
| 2 | Kollidone VA64 | 11.11 | 12.94 |

Extragranular Part

| | | | |
|---|---|---|---|
| 3 | Mannitol | 55.82 | 49.47 |
| 4 | Microcrystalline Cellulose | 20.00 | 22.65 |
| 5 | Crosscarmellose Sodium | 8.33 | 9.71 |
| 6 | Sodium lauryl sulfate | 0.04 | 0.05 |
| 7 | Colloidal silicon dioxide | 0.14 | 0.16 |
| 8 | Magnesium stearate | 0.69 | 0.81 |

Film coating

| | | | |
|---|---|---|---|
| 9 | Opadry film coating | 2.75 | 2.91 |

| | | |
|---|---|---|
| Total | 100.00 | 100.00 |

### The Procedure for Example 5 and 6 Comprises of the Steps:

1. Drug and polymer was sifted through suitable mesh and added into double cone blender and blended for 5 minutes and subsequently processed in HME.
2. The extrudates were milled through suitable screens.
3. Extra granular material was sifted through suitable mesh and blended with step 2 material in double cone blender for 5 minutes.
4. Step 3 lubricated blend was compressed into tablets using compression machine.
5. Compressed tablets then film coated in perforated coating pan

### Examples 7-10

TABLE 6
Pharmaceutical composition Trametinib

| | | Example | | | |
|---|---|---|---|---|---|
| | | 7 | 8 | 9 | 10 |
| | Ingredient Name | % w/w | % w/w | % w/w | % w/w |
| 1 | Trametinib | 1.18 | 1.29 | 1.21 | 1.29 |
| 2 | Kollidone VA64 | 5.88 | 10.00 | — | — |
| 3 | Kollidone K30 | — | — | 6.06 | 6.47 |
| 4 | Mannitol | 59.11 | 55.94 | 57.87 | 55.94 |
| 5 | Microcrystalline Cellulose | 21.18 | 22.65 | 21.82 | 22.65 |

Extragranular Part

| | | | | | |
|---|---|---|---|---|---|
| 6 | Crosscarmellose Sodium | 8.82 | 9.71 | 9.09 | 9.71 |
| 7 | Sodium lauryl sulfate | 0.04 | 0.05 | 0.04 | 0.05 |
| 8 | Colloidal silicon dioxide | 0.15 | 0.16 | 0.15 | 0.16 |
| 9 | Magnesium stearate | 0.74 | 0.81 | 0.76 | 0.81 |

Film Coating

| | | | | | |
|---|---|---|---|---|---|
| 10 | Opadry film coating | 2.91 | 2.91 | 3.00 | 2.91 |

| | | | | |
|---|---|---|---|---|
| Total | 100.00 | 100.00 | 100.00 | 100.00 |

### Examples 11-14

TABLE 7
Pharmaceutical composition Trametinib

| | | Example | | | |
|---|---|---|---|---|---|
| | | 11 | 12 | 13 | 14 |
| | Ingredient Name | % w/w | % w/w | % w/w | % w/w |
| 1 | Trametinib | 1.25 | 1.25 | 1.25 | 1.25 |
| 2 | Kollidone K30 | 7.52 | 8.77 | — | 7.52 |
| 3 | Kollidone VA64 | — | — | 10.65 | — |
| 3 | Mannitol | 59.51 | 23.43 | 20.04 | 59.51 |
| 4 | Microcrystalline Cellulose | 24.68 | 59.51 | 61.01 | 24.74 |

Extragranular Part

| | | | | | |
|---|---|---|---|---|---|
| 5 | Crosscarmellose Sodium | 3.13 | 3.13 | 3.13 | 3.13 |
| 6 | Sodium lauryl sulfate | 0.06 | 0.06 | 0.06 | 0.00 |
| 7 | Colloidal silicon dioxide | 0.16 | 0.16 | 0.16 | 0.16 |
| 8 | Magnesium stearate | 0.78 | 0.78 | 0.78 | 0.78 |

Film Coating

| | | | | | |
|---|---|---|---|---|---|
| 9 | Opadry film coating | 2.91 | 2.91 | 2.91 | 2.91 |

| | | | | |
|---|---|---|---|---|
| Total | 100.00 | 100.00 | 100.00 | 100.00 |

### The Procedure for Examples 7-14

1. Kollidone K30/Kollidon VA64 was dissolved in suitable solvent mixture along with Trametinib and spray granulated onto the substrate (microcrystalline cellulose and mannitol). Obtained granules were collected and dried.
2. Extra granular material was sifted through suitable mesh & used for the lubrication of step 1 granules.
3. Lubricated blend of step 2 was compressed into tablets.
4. Compressed tablets then film coated in perforated coating pan

Examples 5-14 can be prepared by any known methods of solid dispersion i.e. solvent evaporation, HME, Top spray granulation, spray drying, use of hot melt granulation technique. The obtained dried solid dispersion can be further processed to suitable pharmaceutical tablet using known method i.e. direct compression, dry granulation and/or wet granulation method.

### Example 15—Dissolution and Impurity Results

TABLE 7
Results of dissolution studies for Example
5, 7, 9, 11-14 & Mekinist ®

| | Batch No | % DR | | | | | |
|---|---|---|---|---|---|---|---|
| | (pH 4.5 Sodium Acetate | Time points (mins) | | | | | |
| Sr. no. | buffer + 0.75% SLS) | 5 | 10 | 15 | 20 | 30 | 60 |
| 1 | Mekinist ® | 89 | 94 | 96 | 97 | 98 | 99 |
| 2 | Example 5 | 78 | 85 | 92 | 90 | 92 | 95 |
| 3 | Example 7 | 75 | 95 | 97 | 97 | 98 | 99 |
| 5 | Example 9 | 92 | 98 | 100 | 101 | 101 | 102 |
| 6 | Example 11 | 23 | 47 | 67 | 80 | 93 | 94 |
| 7 | Example 12 | 15 | 42 | 62 | 78 | 89 | 91 |
| 8 | Example 13 | 20 | 47 | 70 | 86 | 94 | 95 |
| 9 | Example 14 | 22 | 51 | 71 | 88 | 93 | 94 |

TABLE 8
Impurity data at Initial and 3 month stability

| | | Examples | | | | | | | |
|---|---|---|---|---|---|---|---|---|---|
| | | 7 | 9 | 11 | 12 | 13 | 14 | 11 | 12 |
| | Specifi- | % Impurity | | | | | | | |
| Impurities | cation | Initial | | | | | | 3 Month, ACC | |
| Maximum | NMT | 0.126 | 0.121 | 0.077 | 0.078 | 0.082 | 0.063 | 0.086 | 0.074 |
| Impurity | 1.0% | | | | | | | | |
| Total | NMT | 0.401 | 0.369 | 0.160 | 0.163 | 0.206 | 0.278 | 0.211 | 0.182 |
| Impurity | 1.5% | | | | | | | | |

### Example 16—Bioequivalence Study

An open label, balanced, randomized, single oral dose, crossover, oral bioequivalence study of Trametinib Tablets 2 mg in comparison with Mekinist® (Trametinib) 2 mg tablets (Marketed by Novartis pharma) was performed in healthy, adult, human subjects under fasting conditions.

The outcome of the bioequivalence study for Trametinib compositions as described herein under fasting state complies with pre-defined bioequivalence criteria for Cmax, AUC0-t and AUC0-∞.

We claim:

1. A solid oral pharmaceutical composition comprising a solid dispersion of unsolvated Trametinib or a pharmaceutically acceptable salt thereof and a pharmaceutical acceptable polymer.

2. The solid oral pharmaceutical composition according to claim 1, wherein the polymer is selected from the group consisting of polyvinyl pyrrolidone and derivatives thereof, povidone K-30, Povidone K-60, Povidone K-90, Kollidone K30, Vinylpyrrolidone-vinyl acetate copolymer, Kollidone VA64, Plasdone, Copovidones, Plasdone S630, polyoxyethylene-polyoxypropylene copolymers, polyvinylpyrrolidone vinyl acetate, polyvinylacetal diethylaminoacetate, polyvinyl acetate phthalate, polysorbate 80, polyoxyethylene (40) stearate, polyethylene glycol monomethyl ether, polyethylene glycol, methylcellulose, methacrylic acid copolymer, hydroxypropylmethyl cellulose, hydroxypropyl cellulose, hydroxypropylmethyl cellulose phthalate, hydroxypropylmethyl cellulose acetate succinate, hydroxypropylmethyl cellulose, hydroxypropyl cellulose SSL, hydroxypropyl cellulose SL, hydroxypropyl cellulose L, hydroxyethyl cellulose, soluplus, polyvinyl caprolactam-polyvinyl acetate-polyethylene glycol graft copolymer cellulose acetate phthalate and combinations thereof.

3. The solid oral pharmaceutical composition according to claim 1, wherein the polymer is polyvinyl pyrrolidone and derivatives thereof, copovidone, hydroxypropyl cellulose, hydroxypropylmethyl cellulose phthalate, hydroxypropylmethyl cellulose acetate succinate, hydroxypropylmethyl cellulose and eudragit.

4. The solid oral pharmaceutical composition according to claim 1, wherein the amount of polymer is 0.5 to 50 parts by weight with respect to 1 part by weight of Trametinib.

5. The solid oral pharmaceutical composition according to claim 4, wherein the amount of polymer is 0.5 to 25 parts by weight with respect to 1 part by weight of Trametinib and specifically 0.5 to 10 parts by weight with respect to 1 part by weight of Trametinib.

6. The solid oral pharmaceutical composition according to claim 1, further comprising
0.1-85% w/w of at least one pharmaceutically acceptable diluent,
0.1-20% w/w of at least one pharmaceutically acceptable disintegrant,
0-50% w/w of at least one pharmaceutically acceptable binder,
0-2% w/w of at least one pharmaceutically acceptable surfactant,
0.5-4% w/w of at least one pharmaceutically acceptable lubricant and
0.1-3% w/w of at least one pharmaceutically acceptable glidant based on the total weight of the composition.

7. The solid oral pharmaceutical composition according to claim 1, prepared by a process comprising:
1. preparing the solid dispersion comprising Trametinib and a polymer,
2. further processing solid dispersion with other excipients,

8. The solid dispersion according to claim 7 is prepared by spray drying process, hot-melt process or a fluidized bed process.

9. The solid oral pharmaceutical composition according to claim 7, prepared by a process comprising mixing the solid dispersion with one additive or two or more additives and granulating the mixture, and further comprising a step of tableting granules of the mixture.

10. The solid oral pharmaceutical composition according to claim 1, wherein the pharmaceutical composition is a tablet.
